(19) United States
(12) Patent Application Publication
(10) Pub. No.: US 2005/0176026 A1
(43) Pub. Date: Aug. 11, 2005

(54) Liquid mixing reactor for biochemical assays

(76) Inventors: Carl P. Franck, Ithaca, NY (US); John T. Lis, Ithaca, NY (US); Amber Krause Boehm, Ithaca, NY (US)

Correspondence Address: Michael L. Goldman;Nixon Peabody LLP, Clinton Square, P.O. Box 31051, Rochester, NY, 14603-1051, US

(21) Appl. No.: 10/934,894

(22) Filed: Sep. 3, 2004

Related U.S. Application Data
(60) Provisional application No. 60/500,819, filed on Sep. 5, 2003.

(51) Int. Cl. C12Q 1/68; G01N 33/53

(52) U.S. Cl. 435/6; 435/7.1

(57) ABSTRACT

The present invention relates to methods of reacting a receptor and a target. A reaction liquid having one or more receptors and one or more targets is provided. A confining fluid that is immiscible with the reaction liquid is positioned adjacent a first surface of the reaction liquid. The confining fluid is stirred, thereby allowing the one or more receptors and one or more targets to react with each other. Alternatively, a coverplate is positioned adjacent a first surface of the reaction liquid and reaction between the receptors and targets occurs upon rotating the coverplate. Also disclosed is a system for reacting a receptor and a target. The system involves a holding device having a reaction liquid, a confining fluid adjacent a first surface of the reaction liquid, and a mixing device positioned within the confining fluid. Alternatively, the system can be a substrate, a rotating coverplate, and a reaction liquid between the substrate and the coverplate.

This application claims the benefit of U.S. Provisional Patent Application Ser. No. 60/500,819, filed Sep. 5, 2003, which is hereby incorporated by reference in its entirety.

The subject matter of this application was made with support from the United States Government by the National Science Foundation under award number DMR-0079992. The U.S. Government may have certain rights.

### FIELD OF THE INVENTION

The present invention relates to a liquid mixing reactor for biochemical assays.

### BACKGROUND OF THE INVENTION

Reactions in thin films are important in the modern genetics laboratory. Typically performed under a cover glass, they are found in experimental techniques such as in-situ hybridization, microarrays, and immunohistochemistry (Bowtell et al., “DNA Microarrays: A Molecular Cloning Manual,” Cold Spring Harbor Laboratory Press, Cold Spring Harbor, p. 712 (2003)). One problem associated with thin films in such slide-based techniques is that convective transport processes are absent and reactants diffuse so slowly that assays may require hours or days to reach equilibrium (Duggan et al., “Expression Profiling Using cDNA Microarrays,” Nature Genet. 21:25-32 (1999)). To overcome this diffusion limitation, several researchers have introduced techniques to increase the motion of reactants in slide-based assays (Holloway et al., “Options Available from Start to Finish for Obtaining Data from DNA Microarrays II,” Nature Genet. 32:481-489 (2002)).

DNA microarrays (“DNAM”s) are useful systems for massively parallel identification of DNA fragments. A current research application is to reveal the biochemical circuitry of life at the level of protein-regulated genes by monitoring the expression levels of thousands of genes simultaneously. DNAMs also have great utility for medicine in screening for disease-associated mutations. An important chemical step by which DNAMs operate is the DNA-DNA hybridization reaction. Here, two groups of DNAs are introduced in order to see if complementary sequence matches occur. The first group, the targets, is in an extremely heterogeneous liquid suspension: it consists of DNA clones of all the mRNAs of a particular organism under conditions of interest. These molecules are labeled by means of a fluorescent tag as to their source (e.g., red and green for cancerous and healthy cells, respectively). The second group, the probes, is a library of DNA fragments arrayed as thousands of spots on a solid substrate. These fragments may come from genes whose importance is already recognized as well as from many other candidates whose significance may be assessed by the degree to which they are differentially expressed (e.g., extent to which they differentiate between cancerous and healthy cells). DNAMs suffer from a number of performance and operational limitations, such as fluctuating sensitivity, poor reproducibility, slow throughput, poor calibration, and awkward confinement of the critical reaction volume. These problems are conceivably associated with the above-described hybridization stage of DNAM operations where target DNA in solution is probed for perfect recognition by many spots of DNA on a substrate. The chemical engineering challenge at this stage is to react target solution, a liquid film tens of microns thick spread over square centimeters of substrate area, with the substrate probes in a reasonable amount of time.

Despite intense interest in DNA microarray technology (Thomas et al., Genome Research 11:1227 (2001) and “The Chipping Forecast II,” Nature Genetics Supplement 32:461 (2002)), practical limitations persist in widely used slide-based microarray technology. A solution to the slowness and inefficiency of hybridization reactions has been introduced through flow into gel membranes (Trends in Biotechnology 19:430 (2001)). In addition, an expensive option has been provided by the Nanogen chip (Heller et al., Electrophoresis 21:157 (2000)). However, these technologies sacrifice the speed of batch processing for the improvement of hybridization efficiency with electrical direction of target DNA to one probe spot at a time. Thus, straightforward and convenient improvements to the widely based assays performed on flat solid surfaces (Brown, Nature Gen. 21:33-37 (1999)) are needed. The difficulties associated with conventionally used target-confining coverslips are well known. For example, there is the danger of irreversibly damaging the hybridized spots during removal of the coverslip as a result of dragging the coverslip over the slide at the start of the first wash. In addition, much of the target solution is lost when it flows from underneath the coverslip when the coverslip is first placed on the microarray. None of the currently contending automated hybridization systems reviewed very recently by Holloway et al., Nature Genetics Supplement 32:481 (2002) provide robust techniques for small volume detection.

The Discovery™ system from Ventana Medical Systems, Inc. (Tucson, Ariz.), employs a liquid cover over the target solution, but relatively high volumes of target and a much more complex and expensive air jet stirring system is required. A disadvantage of increased target volume is that it might lead to reduced signal because of target dilution.

An alternative approach to active mixing has been introduced by Advalytix (Brunnthal, Germany) whose ArrayBooster™ system uses surface acoustic waves to agitate volumes as small as 10 μl. However, it is not clear over how wide an area the reactant volume can be spread with this technique.

The present invention is directed to overcoming these deficiencies in the art.

### SUMMARY OF THE INVENTION

One aspect of the present invention relates to a method of reacting a receptor and a target. This method involves providing a reaction liquid having at least one of one or more receptors and one or more targets. A confining fluid that is immiscible with the reaction liquid is positioned adjacent a first surface of the reaction liquid. The confining fluid is stirred, thereby allowing the one or more receptors and one or more targets to react with each other.

Another aspect of the present invention relates to a system for reacting a receptor and a target. The system has a holding device having a reaction liquid which has at least one of one or more receptors and one or more targets. A confining fluid that is immiscible with the reaction liquid is adjacent a first surface of the reaction liquid. The system also has a mixing device positioned within the confining fluid.

A further aspect of the present invention relates to a method of reacting a receptor and a target. This method involves providing a reaction liquid having at least one of one or more receptors and one or more targets. A coverplate is positioned adjacent a first surface of the reaction liquid. The coverplate is rotated, whereby reaction between the one or more receptors and one or more targets occurs.

Yet another aspect of the present invention relates to a system for reacting a receptor and a target. The system has a substrate, a rotating coverslip, and a reaction liquid which has at least one of one or more receptors and one or more targets positioned between the substrate and the rotating coverslip.

One aspect of the present invention relates to a liquid-on-liquid mixing (“LOLM”) method for stirring thin films. This method has the advantage of preserving the small reactant volumes associated with the conventional coverslip method (e.g., 34 μl) while avoiding the potential dangers associated with the use of cover glasses, such as trapped bubbles and scratched slide substrates, and complications such as excessive or insufficient humidification (Best et al., “DNA Microarrays: A Molecular Cloning Manual,” Cold Spring Harbor Laboratory Press, Cold Spring Harbor, pp. 240-256 (2003), which is hereby incorporated by reference in its entirety). This is accomplished by layering a stirrer liquid (i.e., confining fluid), immiscible with and less dense than an aqueous bioreactant solution, over a thin film of the bioreactant solution deposited on a substrate (e.g., a glass slide). This stirrer liquid touches and spreads the reactant solution along the substrate as a cover glass would, but does not contact the substrate surface. Hydrodynamic shear is transmitted across the liquid-liquid interface; by stirring the confining liquid, the fluid in the thin film can be mixed.

The LOLM system of the present invention is distinguishable from the Ventana Medical Systems, Inc. Discovery™ system, in that the LOLM confining fluid (e.g., mineral oil) is thicker and more viscous than Ventana's Liquid Coverslip™, and the LOLM method of the present invention employs a mechanical structure (e.g., a paddle) to stir the mineral oil directly, unlike Ventana's method of directing air jets on the covering liquid. In addition, the LOLM system of the present invention is able to use reactant volumes as small as 10 μl, which can enable researchers to use less material or higher concentrations, potentially obtaining higher signals, compared to the 100 μl or more required for most automated systems (Holloway et al., “Options Available from Start to Finish for Obtaining Data from DNA Microarrays II,” Nature Genet. 32:481-489 (2002), which is hereby incorporated by reference in its entirety), such as the Ventana system.

Compared to both the Ventana and Advalytix systems, the present invention offers the advantages of considerable simplicity and economy.

### BRIEF DESCRIPTION OF THE DRAWINGS

FIG. 1 is a schematic illustration of one embodiment of the liquid-on-liquid mixing system of the present invention. The reaction chamber is bounded from below by a glass slide and on the sides by acrylic. A thin film of reactant solution wets the slide, and a stirring fluid covers the thin film. A stirring paddle, immersed in the stirring fluid, rotates at 3.4 rpm.

FIG. 2 is a schematic illustration of another embodiment of a mixing system in accordance with the present invention in which a coverslip adjacent a reaction liquid is rotated.

FIG. 3 is a flowchart of experimental procedures carried out. Standard incubations, where the antibody solution was spread with a cover glass, were compared with the liquid-on-liquid mixing method of the present invention. In all other steps, the samples received the same treatment.

FIG. 4 is a graph estimating the number of H14 antibody molecules available to any antigen site by diffusion without stirring, as a function of concentration (as a common logarithm of the fraction of the stock 3-5 μM concentration, i.e., −3 denotes a 1:1000 dilution, or 3-5 nM) and time (common log of number of seconds). The straight lines are isopleths representing 10⁶, 10⁷, 10⁸, 10⁹, and 10¹⁰ IgM pentamers in the span of a single-diffusion-length-radius disk. The [symbol] symbols represent conditions where experiments were performed. Next to some of the experimental conditions, other symbols (● clear; ◯ faint, X none) indicate the average quality of the cover-glass-method slides from Table I.

FIG. 5 are photographs showing examples of stained polytene chromosomes with various banding qualities. For each example, a picture with both the Hoechst and rhodamine fluorescence is shown; the grayscale image showing only the rhodamine intensity is evaluated for banding quality. The antibody concentration, incubation time, and incubation method for each picture is shown. Here, the coverslip (unstirred) sample incubated for 10 minutes with the H14 antibody at a 1:500 dilution shows faint banding, while the coverslip (unstirred) sample incubated for an hour at H14 1:10,000 shows no banding. The LOLM (stirred) samples (with the same H14 antibody concentrations and times) both exhibit clear banding. The exposure times were adjusted to compensate for different levels of overall fluorescence. The scale bar in each picture represents 10 μm.

### DETAILED DESCRIPTION OF THE INVENTION

One aspect of the present invention relates to a method of reacting a receptor and a target. This method involves providing a reaction liquid having at least one of one or more receptors and one or more targets. A confining fluid that is immiscible with the reaction liquid is positioned adjacent a first surface of the reaction liquid. The confining fluid is stirred, thereby allowing the one or more receptors and one or more targets to react with each other.

The methods of the present invention relate to improving the reaction rate between two reactants, referred to herein as the target (the molecule of interest) and the receptor (a molecule that interacts with the target as a binding partner).

Suitable targets in accordance with the present invention include, but are not limited to, antigens (e.g., protein antigens), antibodies, haptens, and nucleic acid molecules. A preferred target is a nucleic acid molecule. A more preferred target is a nucleic acid molecule found in an organism selected from the group consisting of bacteria, fungi, viruses, protozoa, parasites, animals (e.g., humans), and plants. Suitable organisms include, but are not limited to, Cryptosporidium parvum, Escherichia coli, Dengue virus, and Human immunodeficiency virus (HIV-1).

Suitable receptors include antibodies, antigens, nucleic acid molecules, aptamers, cell receptors, biotin, streptavidin, and other suitable ligands. When the target is a nucleic acid molecule, the receptor can be a nucleic acid molecule (e.g., capture probe, selected to hybridize with a portion of the target nucleic acid molecule) or another moiety, such as an antibody or other agent capable of binding to and interacting with the target. In a preferred embodiment, the one or more receptors are preferably immobilized on a substrate which is positioned adjacent a second surface of the reaction liquid as described supra. The substrate is preferably a DNA microarray.

Antibodies in accordance with the present invention can be monoclonal or polyclonal or genetically engineered (e.g., single-chain antibodies, catalytic antibodies) and can be prepared by techniques that are well known in the art such as immunization of a host and collection of sera or hybrid cell line technology or by genetic engineering.

The receptor may also be any naturally occurring or synthetic compound that specifically binds the target of interest.

The target may be provided in a test sample. The test sample may be derived from a wide variety of sources, such as physiologic fluids (e.g., saliva, sweat, serum, plasma, urine, tear fluid, spinal fluid, etc.), chemical processing streams, food, waste water, natural waters, soil extracts, etc. Various addenda may be added to adjust the properties of the test sample, depending upon the properties of the other components of the device, as well as on those of the target itself. Examples of solution addenda which may be incorporated into test sample include buffers (to control pH and ionic strength), sample or target solubilizing agents (e.g., nonpolar solvents), and high molecular weight polymers (e.g., Ficoll®, a nonionic synthetic polymer of sucrose, available from Amersham Biosciences (Piscataway, N.J.), and dextran).

In a preferred embodiment, the one or more receptors and the one or more targets are present in the reaction liquid.

A further aspect of the present invention relates to a system for reacting a receptor and a target. The system has a holding device having a reaction liquid which has at least one of one or more receptors and one or more targets. A confining fluid that is immiscible with the reaction liquid is adjacent a first surface of the reaction liquid. The system also has a mixing device positioned within the confining fluid.

The methods and systems of the present invention are particularly suitable for use with oligonucleotide arrays. The art recognizes several approaches to making oligonucleotide arrays. See e.g., Southern et al., “Analyzing and Comparing Nucleic Acid Sequences by Hybridization to Arrays of Oligonucleotides: Evaluation using Experimental Models,” Genomics 13:1008-1017 (1992); Fodor et al., “Multiplexed Biochemical Assays with Biological Chips,” Nature 364:555-556 (1993); Khrapko et al., “A Method for DNA Sequencing by Hybridization with Oligonucleotide Matrix,” J. DNA Seq. Map. 1:375-388 (1991); Van Ness et al., “A Versatile Solid Support System for Oligodeoxynucleoside Probe-based Hybridization Assays,” Nucleic Acids Res. 19:3345-3350 (1991); Zhang et al., “Single-base Mutational Analysis of Cancer and Genetic Diseases Using Membrane Bound Modified Oligonucleotides,” Nucleic Acids Res. 19:3929-3933 (1991); Beattie, “Advances in Genosensor Research,” Clin. Chem. 41(5):700-06 (1995), which are hereby incorporated by reference in their entirety. These approaches may be divided into three categories: (i) Synthesis of oligonucleotides by standard methods and their attachment one at a time in a spatial array; (ii) Photolithographic masking and photochemical deprotection on a silicon chip, to allow for synthesis of short oligonucleotides (Fodor et al., “Multiplexed Biochemical Assays with Biological Chips,” Nature, 364:555-556 (1993) and R. J. Lipshutz et al., “Using Oligonucleotide Probe Arrays To Assess Genetic Diversity,” Biotechniques 19:442-447 (1995), which are hereby incorporated by reference in their entirety); and (iii) Physical masking to allow for synthesis of short oligonucleotides by addition of single bases at the unmasked areas (Southern et al., “Analyzing and Comparing Nucleic Acid Sequences by Hybridization to Arrays of Oligonucleotides: Evaluation Using Experimental Models,” Genomics, 13:1008-1017 (1992); Maskos, et al., “A Study of Oligonucleotide Reassociation Using Large Arrays of Oligonucleotides Synthesised on a Glass Support,” Nucleic Acids Res., 21:4663-4669 (1993), which are hereby incorporated by reference in their entirety).

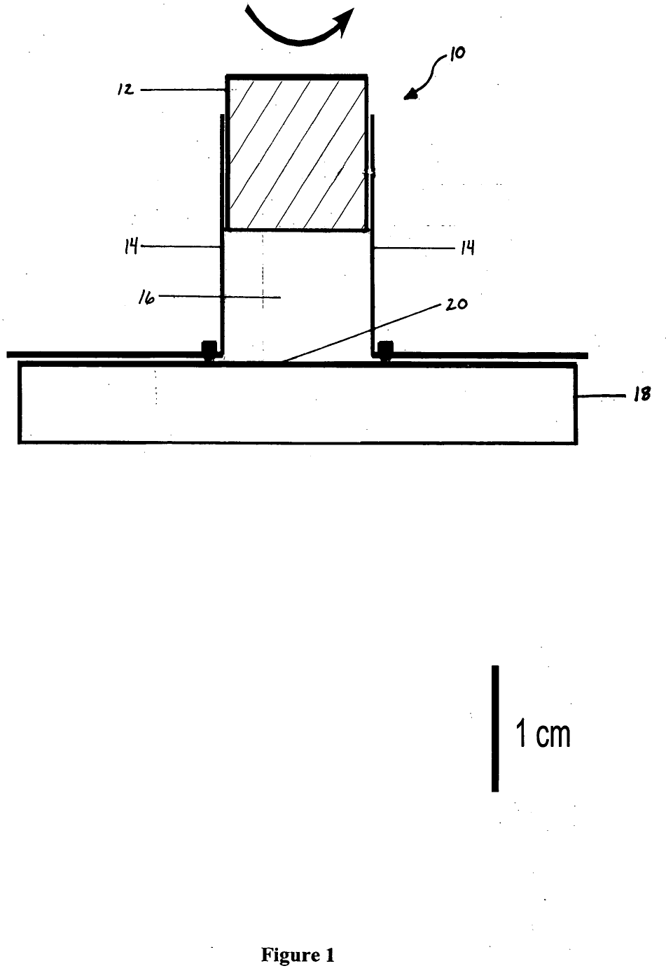

One embodiment of the system of the present invention is illustrated schematically in FIG. 1. Liquid-on-liquid mixing device 10 has holding device 14, which is situated on substrate 18. Substrate 18 may be composed of any of a wide variety of materials, for example, polymers, plastics, ceramics, polysaccharides, silica or silica-based materials, carbon, metals, inorganic glasses, membranes, or composites thereof. In a preferred embodiment, substrate 18 is a glass slide. Holding device 14 is constructed of materials such as acrylic, stainless steel, and anodized aluminum. In practice, a reaction liquid is introduced into holding device 14. The reaction liquid forms thin film 20 on substrate 18. A mixture of receptors and targets may be contained in the reaction liquid. Alternatively, the reaction liquid may contain targets (i.e., a target solution), but no receptors. When the reaction liquid only contains target, receptors are immobilized on substrate 18 prior to introducing that liquid into holding device 14 as described infra. Confining fluid 16 is then introduced into holding device 14. Finally, mixing device 12 is inserted into holding device 14 and brought into contact with confining fluid 16. In operation, movement of mixing device 12 agitates confining fluid 16 and causes mixing in thin film 20 of the reaction liquid, which increases binding between targets and receptors.

Immobilization of receptors on substrate 18 may be carried out by functionalizing substrate 18 with binding members which are attached firmly to the surface of substrate 18. Preferably, the surface functionalities will be reactive groups, such as silanol, olefin, amino, hydroxyl, aldehyde, keto, halo, acyl halide, or carboxyl groups. In some cases, such functionalities preexist on the substrate. For example, silica based materials have silanol groups, polysaccharides have hydroxyl groups, and synthetic polymers can contain a broad range of functional groups, depending on which monomers they are produced from. Alternatively, if the substrate does not contain the desired functional groups, such groups can be coupled onto the substrate by a variety of techniques (e.g., plasma amination, chromic acid oxidation, treatment with a functionalized side chain alkyltrichlorosilane), which are well-known in the art.

Generally, the functional groups serve as starting points for receptors that will ultimately be coupled to substrate 18. These functional groups can be reactive with an organic group that is to be attached to the substrate or can be modified to be reactive with that group, such as through the use of linkers or handles. When employed, the linker molecules are preferably of sufficient length to permit polymers in a completed substrate to interact freely with molecules exposed to the substrate. The linker molecules should be 6-50 atoms long to provide sufficient exposure. The linker molecules may be, for example, aryl acetylene, ethylene glycol oligomers containing 2-10 monomer units, diamines, diacids, amino acids, or combinations thereof.

According to alternative embodiments, the linker molecules are selected based upon their hydrophilic/hydrophobic properties to improve presentation of synthesized polymers to certain receptors. For example, in the case of a hydrophilic receptor, hydrophilic linker molecules will be preferred to permit the receptor to approach more closely the synthesized polymer.

The linker molecules can be attached to the substrate via carbon-carbon bonds using, for example, (poly)tri-fluorochloroethylene surfaces or, preferably, by siloxane bonds (using, for example, glass or silicon oxide surfaces). Siloxane bonds with the surface of the substrate may be formed in one embodiment via reactions of linker molecules bearing trichlorosilyl groups. The linker molecules may optionally be attached in an ordered array, i.e., as parts of the head groups in a polymerized monolayer. In alternative embodiments, the linker molecules are adsorbed to the surface of the substrate.

The reaction liquid employed in the methods and system of the present invention can be a single liquid phase. The reaction liquid preferably has a plurality of reaction liquid droplets, where each reaction liquid droplet has a different receptor or target.

According to the methods and system of the present invention, stirring involves mixing the confining fluid with a mixing device. Preferably, the mixing device has a mixing paddle (as illustrated in FIG. 1), metal disks, ceramic disks, plastic disks, metal cubes, ceramic cubes, plastic cubes, or the like.

For purposes of the present invention, confining fluid includes, without limitation, mineral oil, hexane, heptane, cyclohexane, or any other liquid that forms an immiscible phase with the reaction liquid.

In addition to the above-described embodiments (e.g., relating to microarray technology), the methods and systems of the present invention are applicable to various types of mixing in a fluidic environment. In one embodiment, one of the reactants is immobilized or positioned on a substrate. Besides providing mixing of small volume liquids in the form of thin films, liquid-on-liquid mixing can provide advantages in geometrics of great interest in other contemporary low volume applications. For example, consider a collection of many independent reactions occurring in parallel in small droplet arrays produced by spotting onto a substrate whose wettability is patterned to anchor the droplets. A confining phase of an immiscible liquid which wets the substrate between the spots not only helps stabilize their position, but when stirred, promotes their reactions.

In another embodiment, both reactants are present in a liquid and the method and system of the present invention can be used to move the reactants within the liquid, thereby improving the rate of reaction between the reactants. The reactant liquid can be placed in any suitable holding device.

Another application of liquid-on-liquid mixing in accordance with the present invention addresses the problem of slowness of closed channel mixing in microfluidic systems because of the poor mixing flow afforded by these low Reynolds number systems. Thus far, the best solutions have required either relatively long mixing channels or intricate patterning of channel topography. In either case, extensive microfabrication steps are required. As an alternative, the present invention relates to the use of wide channels that are defined by variability of surface wettability. Not only does confinement by an immiscible liquid overlayer greatly simplify construction of the mixing chamber over chambers buried in a solid, but wider chambers than are readily produced by traditional techniques are possible. Once again, solutions are readily mixed by stirring the confining layer. Fluids are brought in and out of the mixing chamber by means of standard microfabricated holes.

Liquid-on-liquid mixing systems of the present invention are ideally suited to automatic (e.g., robotic) operations. One can replace all manual operations in which one dispensed and exchanged solutions are dispensed in the same chamber by means of small scale pipes, pumps, and valves. For example, in the DNA microarray embodiment, the washing stages subsequent to the hybridization step can be readily accomplished automatically. The removal of the target film is readily accomplished and the first wash begun by simply floating off the confining phase by the addition of the first wash to the chamber. This is in contrast to operations in coverslip based systems, be they passive or actively mixed, where the exchange of the target for the first wash is awkward and requires the delicate removal of the coverslips.

Another aspect of the present invention relates to a method of reacting a receptor and a target. This method involves providing a reaction liquid having at least one of one or more receptors and one or more targets. A coverplate is positioned adjacent a first surface of the reaction liquid. The coverplate is rotated, whereby reaction between the one or more receptors and one or more targets is allowed to occur.

A coverplate is preferably rotated by periodic rotating.

Yet another aspect of the present invention relates to a system for reacting a receptor and a target. The system has a substrate, a rotating coverslip, and a reaction liquid which has at least one of one or more receptors and one or more targets positioned between the substrate and the rotating coverslip.

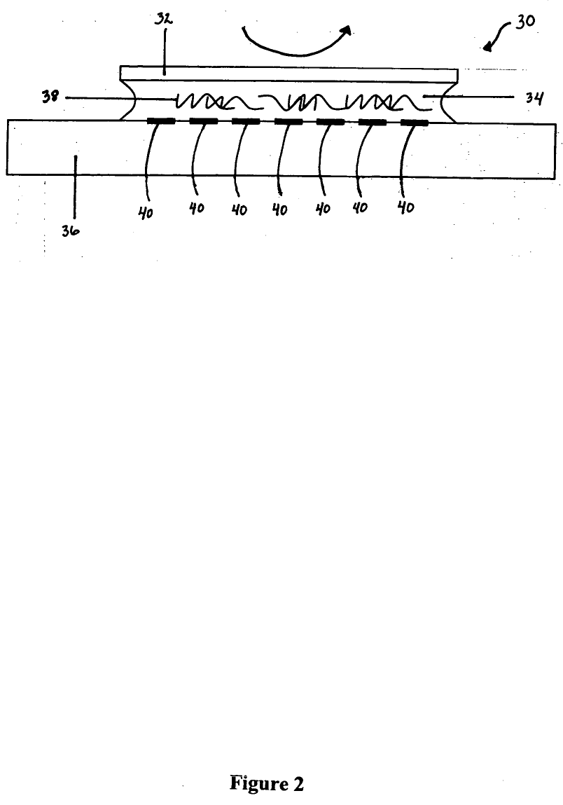

One embodiment of this system of the present invention is illustrated in FIG. 2. Device 30 has substrate 36, upon which is placed reaction liquid 34. Reaction liquid 34 contains targets 38. In one embodiment, as shown in FIG. 2, substrate 36 has receptors 40 immobilized on its surface. Rotating coverplate 32 is placed adjacent reaction liquid 34. In operation, rotating coverplate 32 mixes reaction liquid 34 and thereby facilitates reaction between targets 38 and receptors 40. In an alternative embodiment, reaction liquid 34 contains targets and receptors. Thus, receptors 40 are not immobilized on substrate 36.

The present invention is applicable to procedures and products for determining a wide variety of analytes (i.e., targets), including the following: environmental and food contaminants (e.g., pesticides and toxic industrial chemicals); drugs (e.g., therapeutic drugs and drugs of abuse, hormones, and vitamins); proteins (e.g., enzymes, receptors, and antibodies of all classes); prions; peptides; steroids; bacteria; fungi; viruses; parasites; components or products of bacteria, fungi, viruses, or parasites; aptamers; allergens of all types; products or components of normal or malignant cells; etc. In particular, the following analytes may be detected: T₄; T₃; digoxin; hCG; insulin; theophylline; leutinizing hormones; and organisms causing or associated with various disease states, such as streptococcus pyrogenes (group A), Herpes Simplex I and II, cytomegalovirus, chlamydiae, etc. The present invention may also be used to determine relative antibody affinities and for relative nucleic acid hybridization experiments, restriction enzyme assays with nucleic acids, binding of proteins or other material to nucleic acids, and detection of any nucleic acid sequence in any organism (i.e., prokaryotes and eukaryotes).

### EXAMPLES

The following examples are provided to illustrate embodiments of the present invention but are by no means intended to limit its scope.

### Example 1

### Absolute DNAM Efficiency Measurement

An absolute efficiency measurement of target detection efficiency was performed using DNAMs. Microarrays were constructed consisting of 20 spots each of two genes, Gly-3 and Rubisco, both about 1 kB in size. The target was Gly-3 diluted over four decades. The target was recognized as expected by the appropriate probe gene. The DNA detection efficiency was approximately 0.1% with an uncertainty of less than a factor of 10, completely consistent with the estimate based on bulk diffusion as the rate-limiting process.

### Example 2

### Rotating Coverplate Hybridization System for Stirring Target Solution

In order to overcome this diffusion bottleneck, a pilot mixing experiment was assembled in which the target solution was stirred over the microarray by rotating a coverplate. It was found possible to magnetically suspend and rotate the coverplate by delicately balancing wetting, gravity, and magnetic suspension forces. In doing this, it is important to have a hydration protocol to keep the target solution from drying out as the coverplate was rotated. In order to assemble and remove the coverplate, a magnetic suspension system was developed for getting the microarray in and out for the hybridization chamber without unintentionally shearing the target solution. After many trials, success was obtained by rotating the coverplate for over an hour. As a result, for a microarray of 20 spots laid out along a line, a factor of two variations in hydrodynamic shear rate in a single hybridization experiment was attained.

A water bath thermostat was used to hold the hybridization chamber at the required elevated temperature (65° C.), and control electronics were used to operate a stepping motor drive to rotate the coverplate during hybridization.

### Example 3

### Preliminary Results of Sheared Target Solution Hybridizations

An array of probes 20 spots of the 800 base fragments laid out along a line was used. The target was ssDNA of the same message, prepared by linear PCR. One-hour hybridizations were performed at 65° C. for two target concentrations: 1:15 and 1:1,500 dilutions of the purified linear PCR product. Stirring was carried out in the following manner: 90° rotations over roughly 10 sec. were manually executed every 5 minutes. In the unstirred control, ordinary (non-magnetic) coverslips were used.

Stirred and unstirred arrays were run in pairs at the same time in the thermostat. The result was as follows: 1) The 1:15 dilution yielded the same strong signal for a stirred and unstirred array; 2) The 1:1,500 dilution provided a clearly recognizable signal (the equivalent of a weakly expressed gene) when stirred and an undetectable signal when not stirred. This indicates that stirring at the lower concentration was very effective. By visually comparing spots at different radial distances from the center of the cover-plate (and hence with different shear rates), none the expected dependence of stirring-assisted hybridization on shear rate was observed. These preliminary results were based on only two (one stirred and one unstirred) measurement each for the two target concentrations, i.e. four hybridizations in all.

In a follow-up experiment, the 1 kB Gly-3 and Rubisco gene arrays described supra were used. dsDNA PCR product of Gly-3 was used in the target solution at 1:10, 1:100, 1:1,000, and 1:10,000 dilution of the PCR product (approx. 3 ng/μl). Stirring was carried out continuously at a rate of 0.2 rpm (four times faster than the average rate in the previous experiment). Hybridizations were of 1-hour duration. No stirring enhancement of hybridization signal at the two highest target concentrations was observed. For the 1:1,000 case, a drop in signal with stirring was observed. At the lowest concentration, a signal enhancement with stirring was not detected. These results are preliminary, being based on single hybridizations of each type described.

These results suggest that while stirring does not benefit the strength of hybridization for concentrated targets, very significant improvement is possible with dilute targets if pauses are included in the stirring procedure. No evidence was found that stirring degrades selectivity in DNA microarrays.

### Example 4

### Liquid On Liquid Mixing Demonstration

Experience obtained with the difficulties of operation encountered with conventional DNAMs and the rotating coverplate microreactor described in the previous examples led to the invention of a new means of confining and stirring the target solution: Forming the thin target solution as a wetting layer under an immiscible confining liquid as shown in FIG. 1.

By defining the target film as a wetting layer, the coverslip used in conventional microarrays was eliminated. This eliminates the loss of target solution experienced when the coverslip is placed over the array and the potential damage to the hybridized array when it is removed.

In order to provide uniform stirring of the target layer, the confining liquid is stirred. The liquid-on-liquid mixing technique of the present invention circumvents a critical problem in microfluidics: The dominance of mixing-suppressing laminar flow in thin liquid films. Stirring in a bulk liquid imparts efficient mixing flow patterns that are not available to thin films that are defined by fixed boundaries as in conventional microarrays.

In order to assess the feasibility of a LOLM system for microarray hybridization, a fluid cell and mixer driver was constructed. A mock target solution of standard saline citrate and sodium dodecyl sulfate solutions was prepared at the appropriate concentration. This solution could readily wet a pre-hydrated glass slide that represented the microarray substrate. 28 μl of this mock target solution was used underneath 2 ml of mineral oil as the confining liquid.

In order to follow the progress of the mixing, 0.5 μl of green fluorescent colloidal particles of such a large diameter (1 micron) that their diffusion was insignificant were injected.

In the unstirred system the tracer was essentially unmoved over ten minutes, but in a slowly stirred system the tracer became highly dispersed. At higher speed (0.9 rpm), the dispersal was so complete that the green dye became invisible. This confirms the value of the LOLM scheme.

### Example 5

### Liquid-on-Liquid Mixing Device

The reaction chamber used for the present experiments was a 1.9-cm (¾-inch) diameter hole reamed through acrylic. The glass slide bearing the chromosome sample was held at the bottom opening of the cylinder. An O-ring prevented liquid leakage between the glass-acrylic gap. A 20-μl antibody solution (described infra) was applied to the surface of the slide facing the cylinder, wetting that surface. Three ml of mineral oil (heavy paraffin oil, Fisher) was layered over the thin film of antibody solution. Finally, a paddle (spanning almost the entire diameter of the cylindrical cavity) was immersed in the mineral oil to a depth where its edge was 4 mm from the surface of the slide.

The paddle was turned along the axis of the cylindrical reaction chamber continuously at 3.4 rpm. At the end of stirring, the paddle was lifted out of the mineral oil. The reaction chamber was flooded with distilled water, which lifted the mineral oil away from the slide. The slide was removed from the holder and rinsed with distilled water to remove any residual mineral oil. To avoid contamination, the O-ring was discarded and the reaction chamber was cleaned after each use.

### Example 6

### Comparison of Liquid-on-Liquid Mixing Device with Stationary Coverslip

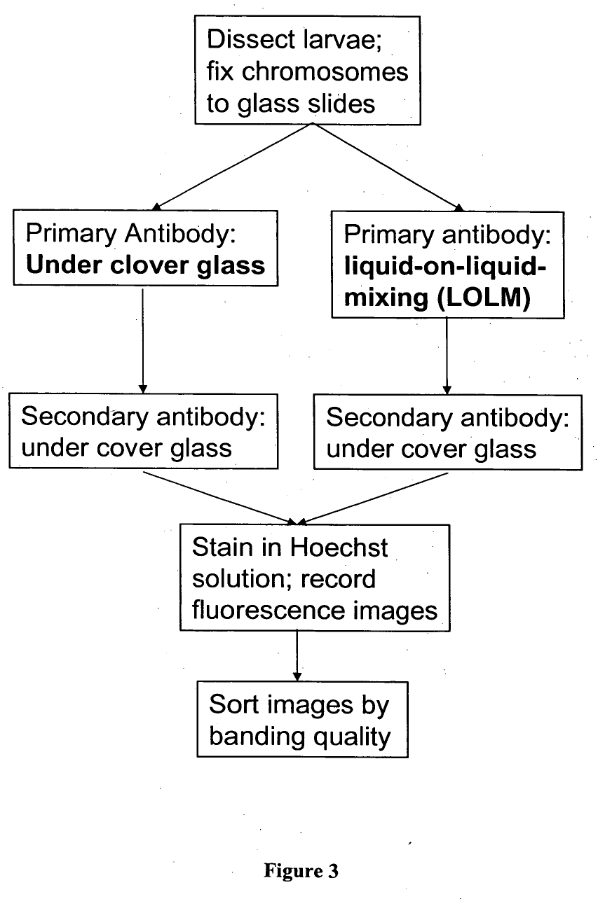

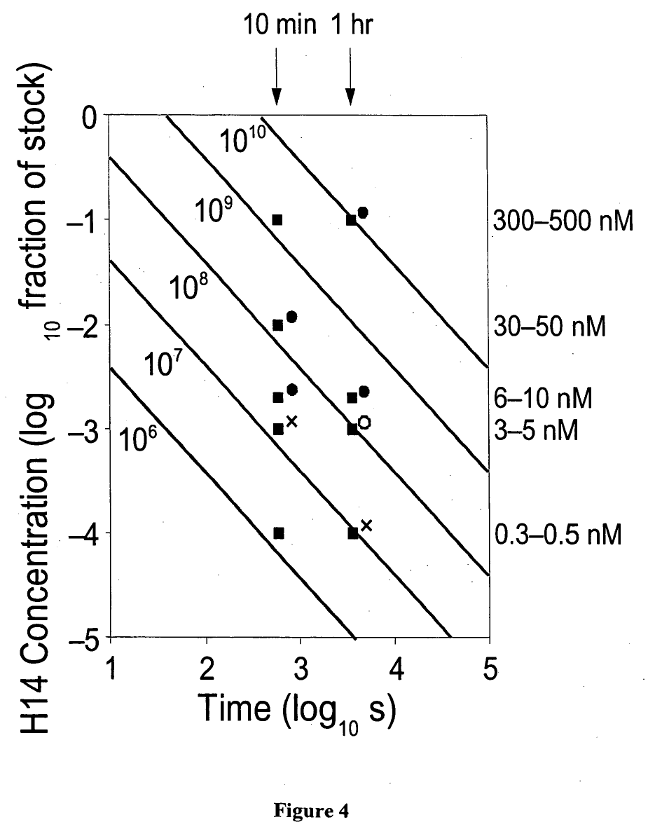

To compare the efficacy of the liquid-on-liquid mixing technique with the conventional coverslip method for spreading thin films of bioreactant solution, parallel immunofluorescence staining experiments that differed in the incubation condition of the primary antibody were performed. This strategy is shown as a flowchart in FIG. 3. To determine the limitations that diffusion places on the system, the experiment was repeated at various antibody concentrations and staining durations, which are shown as points in FIG. 4.

Polytene chromosomes from Drosophila melanogaster third-instar-larval salivary glands were fixed to base-treated microscope slides and stained as described (Lis et al., “P-TEFb Kinase Recruitment and Function at Heat Shock Loci,” Genes Dev. 14:792-803 (2000), which is hereby incorporated by reference in its entirety) with the following modifications. The slides were stained for either 10 minutes or 1 hour with 20 μl of RNA Pol II antibody H14 (MMS-134R supplied as 3-5 mg/ml, Covance Research Products, Berkeley, Calif.) diluted 1:10, 1:100, 1:500, 1:1,000, or 1:10,000 in 5% normal donkey serum (Jackson InmunoResearch Laboratories, Inc., West Grove, Pa.) in 10-mM Tris-buffered saline. The antibody solution was either incubated under a cover glass in a moist chamber or stirred with the LOLM technique. Usually, at least two slides were subjected to each antibody dilution, staining duration, and incubation condition.

The slides were washed, and then secondary antibody stainings were performed by incubation under a cover glass for 1 hour in a moist chamber, using a 1:100 dilution of rhodamine Red-X-conjugated anti-mouse immunoglobulin (Ig) M (Jackson ImmunoResearch Laboratories, Inc.). The slides were then washed again, stained with 0.8 μg/ml Hoechst 33258 (Sigma) in Tris-buffered saline, washed again, and mounted without fluorescent microspheres.

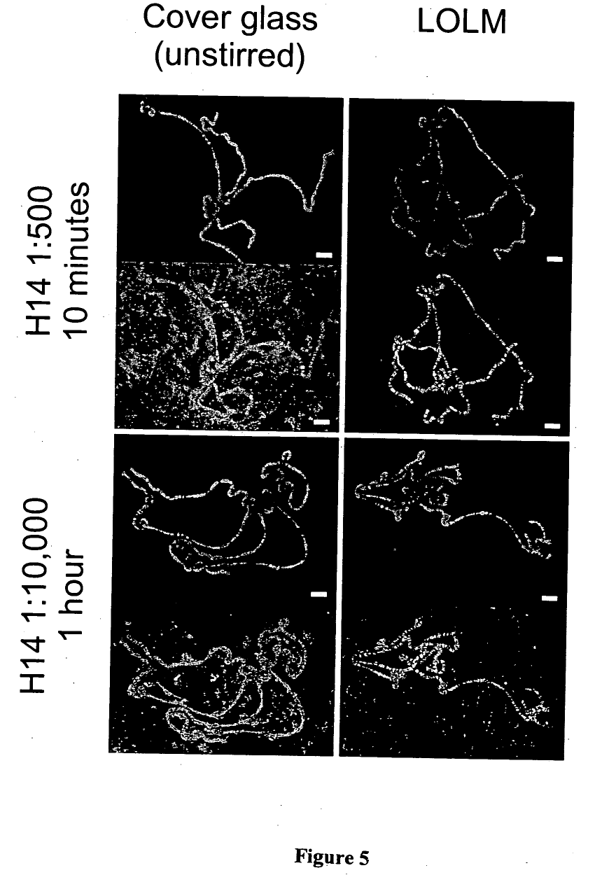

Fluorescent images were recorded, pseudocolored, and overlaid. It was expected that the intensity of the rhodamine (red) fluorescence depends on the quantity of the H14 antibody that binded to the epitopes. The quality of banding in the rhodamine fluorescence for each chromosome image was rated as clear (for distinct banding on most or all of the chromosome arms), faint (for banding patterns with lower fluorescent intensity), or none (for no fluorescence, nonspecific fluorescence, fluorescence patterns consisting only of discrete dots, and incomplete or nonuniform staining). Exemplar images for each banding category appear in FIG. 5.

It is roughly estimated that in all cases, the total binding capacity of the antibody molecules in solution exceeded the total number of epitopes on the slide. However, without stirring, diffusion probably prevented most of the antibodies from visiting and binding to the antigens.

The H14 antibody is an IgM that binds to the heptapeptide repeats phosphorylated at SerS found in the C-terminal domain of elongating RNA polymerase II (Bregman et al., “Transcription-Dependent Redistribution of the Large Subunit of RNA Polymerase II to Discrete Nuclear Domains,” J. Cell Biol. 129:287-298 (1995); O'Brien et al., “Phosphorylation of RNA polymerase II C-Terminal Domain and Transcriptional Elongation,” Nature 370:75-77 (1994), which are hereby incorporated by reference in their entirety). IgMs form pentamers, each with 10 epitope-binding sites and a molecular weight of 950 kDa. The 3-5-mg/ml stock concentration of H14 represents 3-5 μM. At the 1:10,000 dilution (the lowest concentration used) of the primary antibody, there would be 4×10⁹H14 pentamers in the 20 μl antibody solution, which could bind up to 4×10¹⁰ epitopes.

By comparison, the total number of epitopes was estimated as the product of: 10 epitopes per polymerase enzyme (in Drosophila, each RNA polymerase II enzyme has 42 heptapeptide repeats (Patturajan et al., “Growth-Related Changes in Phosphorylation of Yeast RNA Polymerase II,” J. Biol. Chem. 273:4689-4694 (1998); Zehring et al., “The C-terminal Repeat Domain of RNA Polymerase II Largest Subunit is Essential In Vivo But Is Not Required For Accurate Transcription Initiation In Vitro,” Proc. Natl. Acad. Sci. USA 85:3698-3702 (1988), which are hereby incorporated by reference in their entirety), but many fewer than 42 are phosphorylated (Bregman et al., “Transcription-Dependent Redistribution of the Large Subunit of RNA Polymerase II to Discrete Nuclear Domains,” J. Cell. Biol., 129:287-298 (1995), which is hereby incorporated by reference in its entirety); 500-5,000 active polymerase enzymes per chromatid (upon heat-shock induction, 25 Pol II enzymes gather on each of the two hsp70 genes at 87A (O'Brien et al., “RNA Polymerase II Pauses at the 5′ End of the Transcriptionally Induced Drosophila hsp70 Gene,” Mol. Cell. Biol. 11:5285-5290 (1991), which is hereby incorporated by reference in its entirety). Visual estimates of H14 staining suggest that those 50 polymerases represent between 1% and 10% of the signal from the whole squashed nucleus); 1,000-2,000 chromatids per polytene chromosome (1,024 (Alberts et al., Molecular Biology of the Cell, 3^{rd} ed, Garland Publishing, New York, p. 349 (1994), which is hereby incorporated by reference in its entirety); 1,024 (Lewin, Genes VII, Oxford University Press, Inc., New York, p. 558 (2000), which is hereby incorporated by reference in its entirety); 1,000-2,000 (Lodish et al., Molecular Cell Biology, 4th ed, W. H. Freeman and Company, New York, p. 272 (2000), which is hereby incorporated by reference in its entirety); 2,000 (Rudkin et al., in: Beerman, W., (Ed.), Developmental Studies on Giant Chromosomes, Springer-Verlag, New York, pp. 61-85 (1972), which is hereby incorporated by reference in its entirety). In this way, each cell yields 5×10⁶-1×10⁸ epitopes. Each larva's pair of salivary glands consists of about 50 cells, so the total product is some 2×10⁸-5×10⁹ epitopes per slide.

When using a stationary or unstirred coverslip for primary antibody staining, diffusion will limit the number of antibodies that may visit any given antigen. In two dimensions, it is expected that it will take an amount of time:
t=x²/4D
for a particle with diffusion constant D to travel a distance x (Probstein, Physicochemical Hydrodynamics, 2^{nd} ed., John Wiley & Sons, Inc., New York, pp. 120, 123 (1994), which is hereby incorporated by reference in its entirety). The number of antibodies near a single epitope in time t is estimated by calculating the number of H14 molecules present in a disk of radius x centered at the epitope. It is assumed that the antibodies are uniformly distributed under the stationary or unstirred cover glass, with constant area number density:
σ=(Avogadro's number)×(molar concentration)×(reactant solution volume)/(total cover glass area)
barring local depletions and loss of reactant solution, and obtain:
(Number of antibodies in a disk of radius one diffusion−length)=4πD t σ

The diffusion constant of the IgM pentamer is estimated to be 2×10⁻⁷ cm²/s, since the calculation from the Stokes-Einstein fluctuation-dissipation relation (Probstein, Physicochemical Hydrodynamics, 2^{nd} ed., John Wiley & Sons, Inc., New York, pp. 120, 123 (1994), which is hereby incorporated by reference in its entirety) falls within the range expected for molecules of similar mass (van Holde, Physical Biochemistry, 2^{nd} ed, Prentice-Hall, Inc, Englewood Cliffs, N.J., p. 103, Table 4.3 (1985), which is hereby incorporated by reference in its entirety). The cover glasses employed are 22 mm square. Contours in FIG. 4 indicate antibody concentrations and incubation times for which a diffusion-radius disk would be expected to encompass 10⁶, 10⁷, 108, 10⁹, and 10¹⁰ IgM antibody pentamers. Assuming complete and rapid binding, 5×10⁵-1×10⁷ IgM pentamers would be just enough to saturate all 5×10⁶-1×10⁸ epitopes in an isolated squashed nucleus. It is expected that staining conditions on the lower left side of some contour in the range 5×10⁵-1×10⁷ (lower concentrations and shorter times) to starve the epitopes for antibodies and conditions on the upper right side (higher concentrations and longer times) to present enough antibodies in the diffusion-radius disk for all the epitopes.

If the H14 antibodies were monomers instead of pentamers, the molecular diffusion constant would approximately double, which would shift all the contours down and to the left. The factor-of-five increase in number concentration would be balanced by the factor-of-five decrease in the number of binding sites per molecule.

Table 1 shows the image quality of the best-stained polytene chromosome on each slide. Only the best quality stain that could be expected under each set of experimental conditions was reported because varying numbers of images on each slide were taken and any average quality would be less well-defined than the best quality.

TABLE 1
Fluorescent banding quality (● clear; ◯ faint;
X none) of the best image from each slide. Empty categories
denote untested conditions.

| | 10-minute | 10-minute | 1-hour cover | |
|---|---|---|---|---|
| | cover glass | LOLM | glass | 1-hour LOLM |
| H14 dilution | (unstirred) | (stirred) | (unstirred) | (stirred) |
| 1:10 | | ● | ● | |
| 1:100 | ●● | ●●◯ | | |
| 1:500 | ●● | ●●●●◯ | ●●● | |
| 1:1000 | XXX | ●◯◯XXX | ●●◯◯◯ | ●● |
| 1:10000 | | XX | ◯XXX | ●● |

Comparing the banding quality of the samples that underwent incubation using a stationary or unstirred coverslip for 10 minutes and 1 hour, clear images can be obtained with either incubation duration for H14 antibody concentrations as low as 1:500 of stock. At the 1:1,000 antibody concentration with the coverslip method, the 10-minute incubations produced images with very low fluorescence and no banding, but the 1-hour incubations—which allowed an expected six times as many antibody molecules to diffuse to the chromosomes—produced images with clear banding. In FIG. 4, these data are shown against the contours that estimate the number of antibodies in a diffusion-radius disk about each chromosome. These results are in qualitative agreement with the notion that diffusion presents a barrier limiting the quantity of antibody that visits any antigen. Upon closer inspection, two problems emerge. The first is that the threshold between good and bad staining occurs between 10⁷ and 10⁸ IgM pentamers per diffusion-radius disk, which is greater than the prediction that the contour would fall between 5×10⁵−1×10⁷. This is probably due to the simplicity of the diffusion theory. Secondly, when the two points closest to the threshold were compared, the 1-hour 1:1,000 slides had faintly-stained chromosomes, but were expected to see more antibodies than those on the 10-minute 1:500 slides, which had clear staining. Nevertheless, overall, the expected crossover between good and bad staining was seen as a function of antibody concentration and incubation time.

Comparing the banding quality of the samples that underwent incubations using a stationary or unstirred coverslip with those that were stirred with the LOLM method for the same amount of time, it was found that at lower antibody concentrations stirring reduces the fraction of slides with no banding or at best faint images in favor of those with clear images. Good results were obtained for one-hour incubations with LOLM at much lower H14 antibody concentrations (1:10,000, a factor of 20 less) than was required for the static incubations and vastly less than has been used previously (e.g., 1:5 (Lis et al., “P-TEFb Kinase Recruitment and Function at Heat Shock Loci,” Genes Dev. 14:792-803 (2000), which is hereby incorporated by reference in its entirety) with the stationary or unstirred coverslip). This indicates that the liquid-on-liquid mixing technique, where an overlaid, immiscible stirrer fluid transmits good mixing shear into the thin film, can enhance the sensitivity of immunofluorescence staining at low antibody concentrations, where diffusion is limiting.

In the application described herein, the liquid-on-liquid mixing technique of the present invention, has been shown to enhance the sensitivity of immunofluorescence staining. The mineral oil layer also prevents the slide-based experiment from drying out. It is anticipated that the success that this technique has demonstrated delivering antibodies in immunofluorescence staining will carry over to other slide-based assays such as histological preparations and DNA- and antibody-probe arrays.

Although the invention has been described in detail for the purposes of illustration, it is understood that such detail is solely for that purpose, and variations can be made therein by those skilled in the art without departing from the spirit and scope of the invention which is defined by the following claims.

1. A method of reacting a receptor and a target comprising:
providing a reaction liquid comprising at least one of one or more receptors and one or more targets;
positioning a confining fluid that is immiscible with the reaction liquid adjacent a first surface of the reaction liquid; and
stirring the confining fluid thereby allowing the one or more receptors and one or more targets to react with each other.

2. The method according to claim 1, wherein the one or more receptors and one or more targets are present in the reaction liquid.

3. The method according to claim 1, wherein the one or more receptors are immobilized on a substrate which is positioned adjacent a second surface of the reaction liquid.

4. The method according to claim 3, wherein the substrate is a DNA microarray.

5. The method according to claim 1, wherein the one or more receptors are selected from the group consisting of a nucleic acid sequence, an antibody, an antigen, an aptamer, and a cell receptor.

6. The method according to claim 1, wherein the one or more targets are selected from the group consisting of a nucleic acid sequence, an antibody, an antigen, and a hapten.

7. The method according to claim 1, wherein the reaction liquid comprises a single liquid phase.

8. The method according to claim 1, wherein the reaction liquid comprises a plurality of reaction liquid droplets.

9. The method according to claim 8, wherein each reaction liquid droplet comprises a different receptor or target.

10. The method according to claim 1, wherein said stirring is carried out with a mixing device.

11. The method according to claim 10, wherein the mixing device comprises one or more of a mixing paddle, metal disks, ceramic disks, plastic disks, metal cubes, ceramic cubes, or plastic cubes.

12. The method according to claim 1, wherein the confining fluid is a mineral oil, hexane, heptane, cyclohexane, or any other liquid that forms an immiscible phase with the reaction liquid.

13. A system for reacting a receptor and a target comprising:
a holding device comprising a reaction liquid which comprises at least one of one or more receptors and one or more targets;
a confining fluid that is immiscible with the reaction liquid adjacent a first surface of the reaction liquid; and
a mixing device positioned within the confining fluid.

14. The system according to claim 13, wherein the one or more receptors and one or more targets are present in the reaction liquid.

15. The system according to claim 13, wherein the one or more receptors are immobilized on a substrate which is positioned adjacent a second surface of the reaction liquid.

16. The system according to claim 15, wherein the substrate is a DNA microarray.

17. The system according to claim 13, wherein the one or more receptors are selected from the group consisting of a nucleic acid sequence, an antibody, an antigen, an aptamer, and a cell receptor.

18. The system according to claim 13, wherein the one or more targets are selected from the group consisting of a nucleic acid sequence, an antibody, an antigen, and a hapten.

19. The system according to claim 13, wherein the reaction liquid comprises a single liquid phase.

20. The system according to claim 13, wherein the reaction liquid comprises a plurality of reaction liquid droplets.

21. The system according to claim 20, wherein each reaction liquid droplet comprises a different receptor or target.

22. The system according to claim 13, wherein the mixing device comprises one or more of a mixing paddle, metal disks, ceramic disks, plastic disks, metal cubes, ceramic cubes, or plastic cubes.

23. The system according to claim 13, wherein the confining fluid is a mineral oil, hexane, heptane, cyclohexane, or any other liquid that forms an immiscible phase with the reaction liquid.

24. A method of reacting a receptor and a target comprising:
providing a reaction liquid comprising at least one of one or more receptors and one or more targets;
positioning a coverplate adjacent a first surface of the reaction liquid; and
rotating the coverplate, whereby reaction between the one or more receptors and one or more targets occurs.

25. The method according to claim 24, wherein said rotating is periodic.

26. A system for reacting a receptor and a target comprising:
a substrate;
a rotating coverslip; and
a reaction liquid which comprises at least one of one or one or more receptors and one or more targets positioned between the substrate and the rotating coverslip.

27. The system according to claim 26, wherein said rotating is periodic.
